(19) United States
(12) Patent Application Publication
(10) Pub. No.: US 2006/0094975 A1
(43) Pub. Date: May 4, 2006

(54) Non-invasive sensor to analyze visually the level of muscle activity

(76) Inventor: Mario Manto, (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/251,108

(22) Filed: Oct. 14, 2005

Related U.S. Application Data
(63) Continuation-in-part of application No. PCT/BE04/00054, filed on Apr. 16, 2004.
(60) Provisional application No. 60/463,897, filed on Apr. 16, 2003.

(51) Int. Cl. A61B 5/04

(52) U.S. Cl. 600/546

(57) ABSTRACT

Embodiments of a sensing device for measuring muscle activity comprise an interface with electrodes, an active amplifier, and an electronic circuit in connection with a display, said display comprising at least one semiconducting polymer LED.

### RELATED APPLICATIONS

This application is a continuation-in-part of PCT Application No. PCT/BE2004/000054, filed on Apr. 16, 2004, which claims priority to U.S. Provisional Application No. 60/463,897, filed on Apr. 16, 2003, the contents of both of which are hereby incorporated by reference.

### FIELD OF THE INVENTION

Certain embodiments of the present invention concern non-invasive, lightweight, flexible, ergonomic, aesthetic and portable tools to analyze the level of muscle activity visually.

### BACKGROUND

Classical study of muscle activity in human requires the use of cables linked to an acquisition unit. EMG activity is recorded either using needle electrodes/wires inserted in the muscle, either using surface EMG electrodes. So far, there is no technique available to estimate visually the voluntary (such as a movement of the hand or the neck) or involuntary (such as tremor or dystonia) muscle activity with color codes, taking into account ergonomics (a tool which would be lightweight, portable and without cables), and non-invasively. Such a tool would be of great help and benefit for the non-invasive follow-up of patients and for the diagnosis of neuromuscular diseases. For instance, the diagnosis of diseases like torticolis, hand dystonia, upper limb tremor would be easier.

Patent application WO00/51543 relates to a muscle activity monitoring system comprising a sensor, a processor, a display, a power source and a housing. This system suffers from various drawbacks. The markers constituting the display undergo a permanent color change. Moreover, the display is non-erasable and single-use. The power source is a battery, which is irreversably activated. Further it only allows the display of very low quantities of information.

U.S. Pat. No. 6,002,957 relates to an EMG electrode support belt comprising a 9×7 array of electrically conductive electrodes. The measurements need to be evaluated by a physician. The device has several drawbacks like its predetermined pattern of rows and columns and high power consumption.

In U.S. Pat. No. 5,058,602 a method of EMG scanning muscles is disclosed. However, the scanner used has a bandwidth of only 100 to 200 Hz. The display is flexible nor extensible. Also here an examiner is required to carry out the method.

### SUMMARY OF CERTAIN INVENTIVE ASPECTS

Certain embodiments of the present invention aim to provide a non-invasive, stretchable, lightweight, flexible, ergonomic and portable sensing device for measuring muscle activity.

Certain embodiments relate to a sensing device for measuring muscular activity comprising an interface for connecting with a muscle to be measured and a display (video display and/or digital display). In one embodiment, the display comprises at least one semiconducting polymer LED and arranged for receiving a signal comprising information on the muscular activity from said means for establishing a connection.

In one embodiment the interface comprises electrodes, an active amplifier and an electronic circuit in connection with the display. Further the interface preferably comprises fine wire electrodes. In an alternative embodiment the interface comprises at least one membrane and means for circulating a liquid connected to the membrane. Optionally the interface further comprises electrodes, an active amplifier and an electronic circuit in such embodiment. The means for circulating liquid is typically a pump.

In one embodiment, the active amplifier is advantageously provided with a battery. The electronic circuit preferably comprises a passive filter.

In one embodiment, the semi-conducting polymer LED comprises a substrate, an epoxy membrane, an anode, a conducting polymer, an emissive polymer layer, a cathode.

In an advantageous embodiment the sensing device comprises at least two polymer LEDs and an inverter is arranged between the electronic circuit and one of the polymer LEDs. Optionally an analogue converter is arranged between the electronic circuit and the display.

In one embodiment the sensing device is portable by a patient. The connection between the electronic circuit and the display may advantageously be a wireless connection.

In a specific embodiment the sensing device further comprises an electrode for measuring nerve activity.

In an alternative embodiment the sensing device comprises a stack of polymers LEDs, two consecutive polymer LEDs of the stack being separated from each other with substrate that prevents light to pass.

In yet a further embodiment the sensing device further comprises a waterproof covering.

The sensing device may further be provided with a plurality of electrodes, whereby the electrodes are positioned in such arrangement that information on the direction of muscular activity is derivable.

In one embodiment the substrate is elastomer.

In another specific embodiment the sensing device further comprises at least one reservoir.

In yet another embodiment the polymer LED comprises an enzyme (nitrate reductase) for detecting nitric oxyde.

Advantageously, the electrodes may be fixed to bearings.

In another aspect certain embodiments relate to an injection needle comprising the sensing device as previously described.

In a further aspect certain embodiments relate to a method for measuring muscle activity of a patient comprising the step of maintaining the interface with electrodes of the sensing device as described upon the patient's skin for a sufficient time to obtain a measure and, possibly, a recording of muscle activity of the patient.

### SHORT DESCRIPTION OF THE DRAWINGS

FIG. 1 represents an embodiment of a color-EMG sensor.

FIG. 2 represents a video display suitable for two colors.

FIG. 3 represents a digital display.

FIG. 4A represents a more detailed view of an embodiment of a color-EMG.

FIG. 4B represents an embodiment of a bio-color-EMG.

FIG. 5 represents a detailed view of the color-code provided on the polymer LED.

FIG. 6 represents a color-EMG arranged for comparing the contribution of various nerve fibres to the (innervation) of some muscles.

FIG. 7 represents a color-EMG arranged for indicating both the intensity and the direction of muscular activity.

FIG. 8 represents some examples of a color-EMG as in FIG. 7 in use.

FIG. 9 represents a color-EMG to replace a deficit of skin.

### DETAILED DESCRIPTION OF VARIOUS EMBODIMENTS

In one embodiment, the sensor combines the use of active differential electromyographic electrodes fixed directly on the skin (2) such as a Delsys electrode (www.delsys.com/products/electrodes.htm) coupled to digital video display coupled to semi-conducting polymer LEDs (7). Semi-conducting polymer LEDs are electro-luminescent polymers such as the one described by Braun D., Semi-conducting polymer LEDs. Materials today. June 2002; Elsevier Science, pp. 32-39. These polymers LEDs (7) are flexible and present switch on and off characteristics suitable for video display (10) applications.

Throughout the description the term ‘polymer LED’ is used to denote a light-emitting diode made of semiconducting organic polymers. The term OLED (Organic LED) may be considered as a synonym, as the word ‘polymer’ in ‘polymer LED’ refers to the polymer included in an OLED (often derivatives of PPV—poly(p-phenylene vinylene)—are used as the polymer dyes in OLEDs). The term ‘polymer LED’ may equally be understood as a light-emitting polymer. Among the advantageous features of polymer LEDs especially the reduced power consumption and the fact that they can be applied in portable devices are worthwhile mentioning.

Thanks to the development of active EMG electrodes, the signal-to-noise ratio is improved. These electrodes are directly fixed to the skin (2) of a patient. The signal is amplified directly on the skin (2) and filters can be implemented in the amplifier.

Enclosed FIGS. 1 to 3 show the characteristics of an embodiment of the (non-invasive, lightweight, ergonomic and portable by the patient) sensor, which can be applied directly upon the skin (2) of a patient in order to provide information on the level of muscle (1) patient activity. The EMG electrode comprises an electrode interface (3), an active amplifier (4), a battery (5), an electronic circuit (6) connected to a semi-conducting polymer LED (7). The semi-conducing polymer LED (7) is advantageously a flexible, light-weight, extensible polymer, adaptable to the body forms and which may change its calorimetric characteristics. Furthermore, this polymer requires less energy for a color modification than other displays.

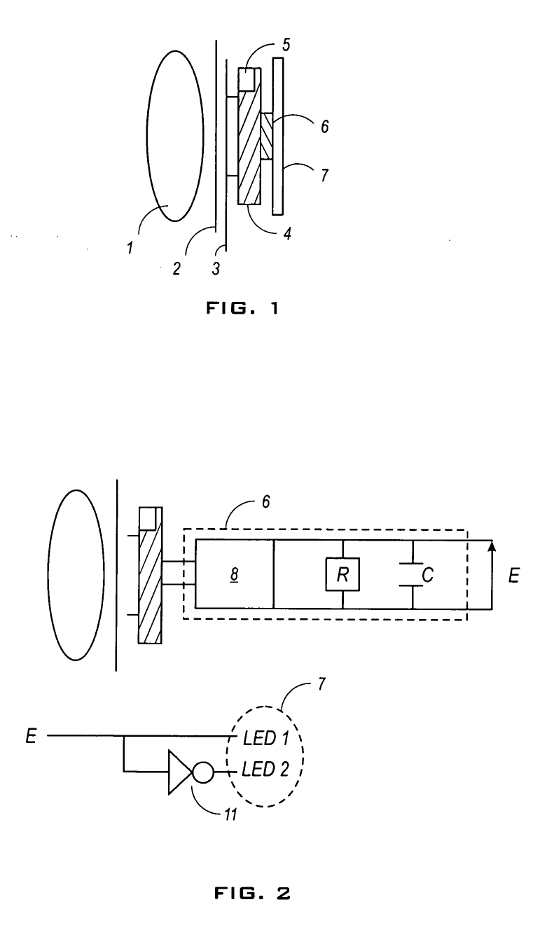

In FIG. 2 is presented the basic principle of the sensor presenting a video display for two colors. The EMG signal is therefore amplified and filtered. A rectifier (8) or inverter (11) can also be used.

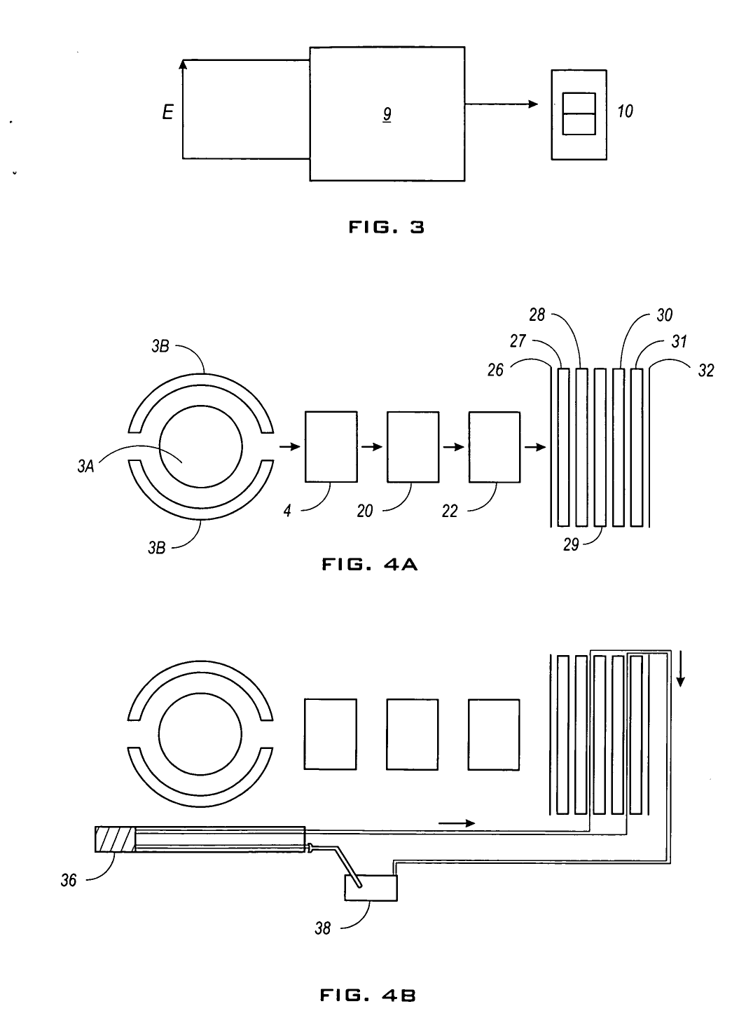

FIG. 3 presents an alternate embodiment of the color-EMG sensor, which comprises a digital display (10) of the EMG activity. The numbers will appear according to the level of EMG activity. Said system also comprises a classical analogue converter (9) for a direct visual display.

Certain embodiments of the sensor present the following advantageous characteristics.

The color-EMG sensor of certain embodiments is fixed on the skin of the neck in patients suffering from torticolis. The sensor informs the observer of the level of muscle hyperactivity (the color selected is dependent on the level of EMG activity). This procedure will improve the diagnosis, leading advantageously to a drug administration in the early stages of the disorder.

Two units of an embodiment of a color-EMG sensor are fixed respectively on the flexor carpi radialis muscle and extensor carpi radialis muscle in a patient suffering from a upper limb tremor. The LEDs flash asynchronously in Parkinson's disease, whereas they flash synchronously in Essential Tremor. Furthermore, the level of EMG activity of each muscle will appear thanks to the color flashing. Therefore, this technique can be used non-invasively or invasively, with the introduction of two fine wire electrodes in the muscle of the patient by the general practitioner.

Certain embodiments allow an analysis of the activity of muscle groups during clinical examination. Several color-EMG sensors are fixed on the patient's lower limbs at the level of the thighs and the legs. This technique will help the neurologist to identify muscles, which are overactive and those that are under-active during gait.

Furthermore, this sensor allows a diagnosis of primary orthostatic tremor (POT). Certain embodiments of the color-EMG sensor may flash at a high frequency (13 to 18 Hz) if fixed at the level of weight-bearing muscles while the patient is standing (this disorder is characterized by high-frequency synchronous discharges at a frequency of 13 to 18 Hz).

The sensor can be used for the detection of myoclonus. Embodiments of the color-EMG sensor will detect the brief and involuntary contractions (usually with a duration of less than 150 msec) by flashing on the skin.

A “dream” for the rehabilitation specialists is to estimate visually and preferably non-invasively the activity of the muscle groups (agonists/antagonists/synergic) during rehabilitation. Embodiments of the color-EMG will help in this task by informing which muscle is active as compared to the other ones.

In addition, embodiments of the sensor allow a non-invasive analysis of muscle activity following a hand grafting. These patients need to take immunosuppressive drugs. Therefore, needles are usually avoided. Embodiments of the color-EMG sensor will help the therapist to follow the recovery.

Embodiments of the sensor can be used for analysis of EMG activity non-invasively on babies and on children, especially in intensive care units.

The use of the color-EMG sensor is also proposed in sports to estimate the level of contraction.

For a research perspective, the sensor comprises fine wire electrodes (inserted in the muscle), the color-EMG sensor will be used to analyze the muscle activity in neuromuscular diseases. Advantageously, the analysis of the patient's muscle activity will be improved with embodiments of the sensor because the signal-to-noise ratio is increased compared to a known technique that requires long connection means between a sensor and a recording apparatus.

A further aspect of certain embodiments is related to a method for measuring muscle activity of the patient, which comprises the step of maintaining the interface with electrodes of an embodiment of the sensing device upon the patient's skin for a sufficient time to obtain a measure and, possibly a recording of muscle activity of the patient.

Using several groups of wires inserted in the muscle or using multi-channels needles, the color-EMG will allow to analyze simultaneously the activity of distinct groups of muscle fibers, to detect overactivity or underactivity of different portions of the muscle.

In a basic application an embodiment of a color-EMG sensing device is placed e.g. on the forearm of a patient suffering from involuntary rhythmic contractions of the upper member. A color-code is defined for the intensity peak of the contraction. For example, if the maximum intensity is 0.5 mV, this amplitude is given a red code. A second code is attributed for indicating the average contraction frequency of the muscle. For example, an average frequency of 5 Hz is given a blue code. By providing e.g. 10 distinct intensity (and/or frequency) levels with different colors, one obtains a device suited for monitoring the fluctuations in the muscle contractions.

In several applications the person skilled in the art can use different sensors suitable for different parts of a body or for different patient's bodies. Embodiments of the sensor allow detecting easily and directly the contraction of several muscles. Among other things, it is possible to detect hypoactive and hyperactive regions of muscles. Such hyperactive contraction of a muscle could be detected even after an activity when the muscle is not able to relax.

Furthermore, this color-EMG sensor could be used in order to find out whether two muscles have a synchronous or asynchronous activity. For a neurologic examination, it allows detecting in a non-invasive way myotatic reflex activity of a muscle. One is also capable of distinguishing between different muscle spasms (slow development or rapid development).

Furthermore, embodiments of the color-EMG sensors could be used for muscular rehabilitation of a patient. Similar EMG sensors are put upon several body areas of a patient, e.g. one not affected and another body area, which needs rehabilitation. The patient can therefore control the contraction of a muscle. Furthermore, this feature allows the medical staff to control rehabilitation of several patients at the same time. This non-invasive technique allows one therapist to guide simultaneously several patients during a training procedure or during rehabilitation.

Furthermore, a wireless connection system of the sensor with a personal computer allows a third person to control remotely the physical activity of the different patients.

With several sensors upon different parts of a patient's body, it is possible to create a dynamic mosaic of different colors with different sensors. Such a mosaic of different colors also has an aesthetic aspect. The color-EMG can take the shape of a mask, allowing the identification of muscles involved in facial emotions.

The color-EMG(s) can be integrated for example in a collar and convey to the patient information on the activity of a certain muscle. Its lightweight, extensible and flexible characteristics allow its use in clothes, including bathing suit and underwear water swimming set (neoprene wetsuit). It may also be placed in a jewel, a watch, earrings and so on. Furthermore, during some periods of the day, e.g. when the person is in company, the display could be regulated such that it adopts the color of the clothes in order to remain as unnoted as possible, thus also respecting the patient's privacy.

FIG. 4A represents with more details than in FIG. 1 the features of an embodiment of a color-EMG sensor comprising a ground electrode (3A), electrodes (3B), an active preamplifier (4), filters (20), a micro-controller (22) (electronic circuit comprising an analogue/digital convertor (ADC)) and connected to the semi-conducting polymers LED. Said polymer LED comprises a substrate (26), an epoxy membrane (27), an anode (28), a conducting polymer (29), an emissive polymer layer (30), a cathode (31) and a cover (32).

The sensor can be improved as proposed in FIG. 4B, by providing it with a membrane to be inserted into the muscle. When the membrane of a muscle cell is disrupted, substances (like e.g. lactic acid) are released, which cause a drop of the pH. This extra-cellular liquid obtained from the muscle is transported to the semi-conducting polymer LED and will modify its color according to the pH change in the muscle (due to the interaction with the polymer). For transporting the liquid the membrane (36) is connected to a means for circulating the fluid, like e.g. micro-pump (38). In this case the resulting color on the semi-conducting polymer LED display depends not only on the electrical activity produced by the muscle, but also on the intramuscular pH measured by membrane (36). The color-EMG is thus turned into a so-called bio-color-EMG.

Furthermore, as shown in FIG. 4B, a feedback loop can be integrated into the device. A positive feedback loop is obtained when the flow rate of the pump (38) is increased as the electric activity of the muscle increases. This reduces the liquid exchange on the membrane because the time available for the exchange is shortened. This way a bio-color-EMG is obtained wherein the selection of the polymer LED colors is mainly determined by the signal amplification. By reducing the flow rate of the pump (38) when the electric activity of the muscle is increased, a negative feedback loop can be achieved. In this way metabolites (like lactic acid) are added to the circulating liquid, which strongly affects the color of the polymer LED. One thus disposes of a bio-color-EMG wherein the electric signal is of less importance to determine the colors.

In a specific embodiment the sensing device can be provided with more than one membrane as previously described. One can then for example use two membranes for a same muscle, which allows getting a better idea of how the lactic acid is spread in the muscle.

Another advantageous feature of the bio-color-EMG is that it can operate in a loop. The liquid that has been transferred to the polymer LED can be reinjected in the loop by means of pump (38).

Further features of the color-EMG are described subsequently. It should be noted that the same features can also be attributed to the bio-color-EMG as presented above.

FIG. 5 represents an illustration of the color code. It shows a polymer LED (51) with color code (52). The EMG intensities are ordered by color, e.g from blue to red. The code takes into account a preselected duration of the period wherein the electric signal is analysed (with horizontal bar 54). A cursor (53) is provided which moves over the color code (52) to indicate in real time the instantaneous intensity of the contraction or the average value over of a certain period of time. The color code also allows distinguishing between a myoclony (very short muscular activity) and a prolonged muscular activity (like in certain dystonies or cramps). The color-code is autocalibrated: the color-EMG permanently monitors the minimum and maximum electric activity in order to adapt its color codes.

By coupling the color-EMG with a micro-electrode measuring the activity of the nerve innervating the muscle one can obtain on the same flexible display a color code that simultaneously indicates the activity of both the nerve and the muscle. This very concept can be extended to a flexible tab that fits the body shape and provides simultaneous information on the nerve and muscular activity.

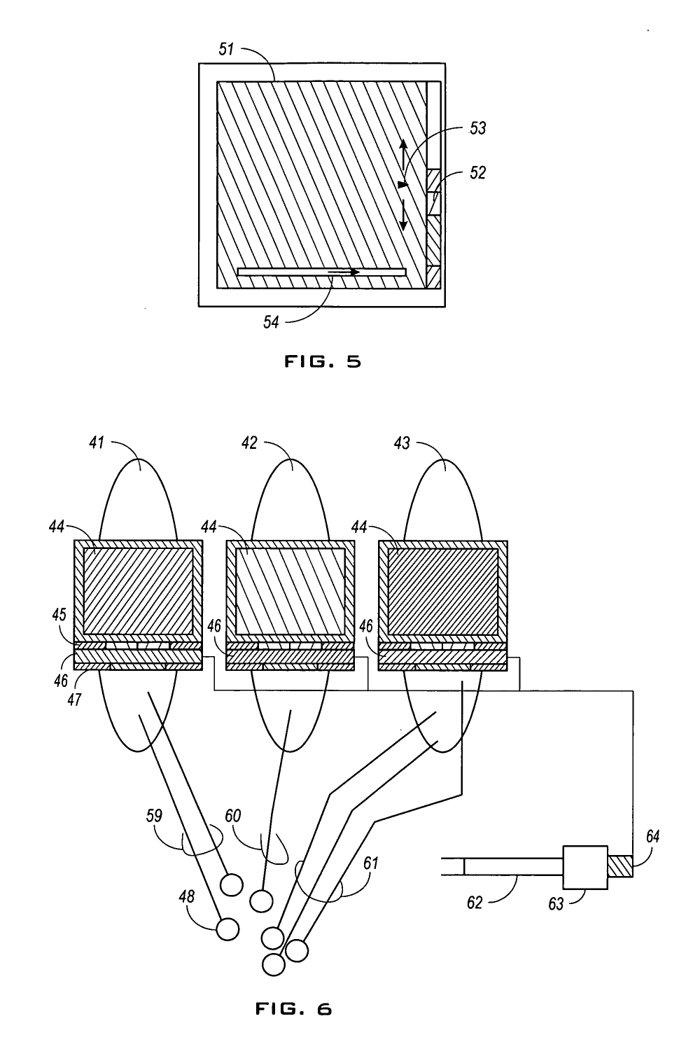

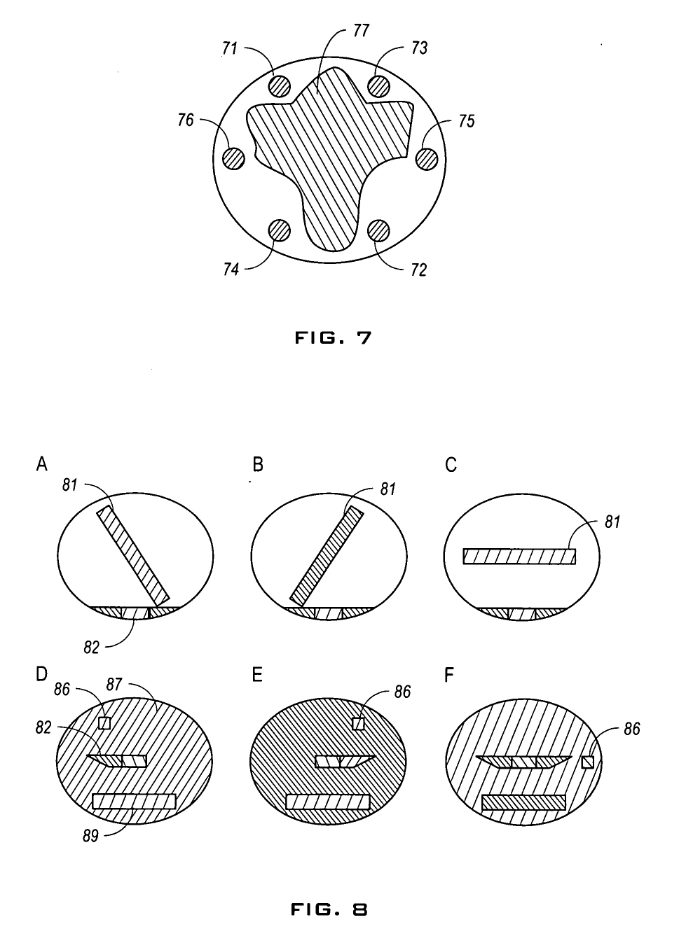

In another application the color-EMG can be used to compare the nerve fibres and the innervation of several muscles dependent on a same nerve. An illustration is provided in FIG. 6. Suppose three muscles (41,42,43) depend on a same motor nerve. Each nerve comprises a multitude of axons. The axons are grouped into nerve fascicles (or groups of axons). While exploring the axons activity by means of a micro-electrode (62), it is very useful to know to which extent a certain zone of the nerve (59,60,61) contributes to the muscular activity (in FIG. 6 measured by 3 color-EMGs). The micro-electrode is coupled to a pre-amplifier (63) connected to an A/D converter and a micro-controller (64). The micro-controller (64) is connected to the three polymer LEDs that convert the nerve activity into color codes. When the micro-electrode approaches zone (59), the polymer LED is green, for zone (60) it is blue and for zone (61) it is red. This means that for a given muscular activity zone (59) contribute on a medium level to the innervation of muscle (41), for zone (60) there is little contribution, whereas for zone (61) contributes to a large extent to the activity of muscle (43). In FIG. 6 polymer LEDs (44) and (46) indicate the activity of muscles with color code (45) and nerves with color code (47), respectively.

As opposed to classical LEDs, polymer LEDs can also be manufactured in a multi-layer version. A layer can be thrown away after use during some time or when the display has been damaged, while one can still benefit from the underlying active amplification. Between two layers of the polymer LED a substrate is placed that prevents light from passing towards the lower layers.

Another advantageous feature of the portable color-EMG is that it can easily be adapted to the shape of the muscle, which renders the use of the color-EMG extremely flexible. As an example, it can be made to fit the shape of the upper lip or of the nostril. As a further example, a color-EMG also can be made to fit the shape of the tongue. In order to analyze the activity of the tongue on both sides of the tongue a color-EMG with a plurality of polymer LEDs can advantageously be used.

Another interesting application field is the detection/monitoring of muscular activity in underwater conditions. The color-EMG can then be used to indicate whether a muscle operates normally, whether cramps or paralysis appear or when a sudden muscular weakness occurs due to an intense effort. For such applications the sensing device is integrated into the mask of the diver, for example. The same concept can be applied to a diver monitoring other divers in his neighbourhood.

The color-EMG can also be arranged for detecting the instantaneous direction of the maximum muscular activity as well as the intensity of the contraction. FIG. 7 represents a bottom view of an embodiment of such a color-EMG. Two electrodes (71,72) are provided for a first channel, two (73,74) for a second and two (75,76) for a third one. A connection (77) to the ground is provided as well. This arrangement allows determining at each time instant whether the difference in potential is maximum for the first, second or third channel. The channel with maximum activity can be indicated by the polymer LED by its direction for example with a bar (81) (see FIG. 8). In FIG. 8A channel 1 has the highest intensity, said intensity being between 67% and 100% of the maximum intensity (as can be read from color-code 82). In FIG. 8B channel 2 has the highest intensity among the three channels, being between 0 and 33%. FIG. 8C shows a case where channel 3 has the highest intensity (between 34% and 66% of the maximum). An alternative is shown in FIG. 8D-F. There the display shows a small square (86) indicating the channel having the highest intensity. Combined with the intensity indication (87) (just as explained previously), one can thus obtain information on both direction and intensity. Optionally the polymer LED can also give an indication of the average activity over a certain period of time. This is indicate by the horizontal bar (89) in FIG. 8D-F.

By using an appropriate elastomer like a siloxane (methylsiloxane, polysiloxane) as the polymer LED's substrate, one can obtain a stretchable color-EMG. Such elastomers are well tolerated by the human body, are light and do not provoke allergic reactions. By using an elastomer the device also becomes more flexible.

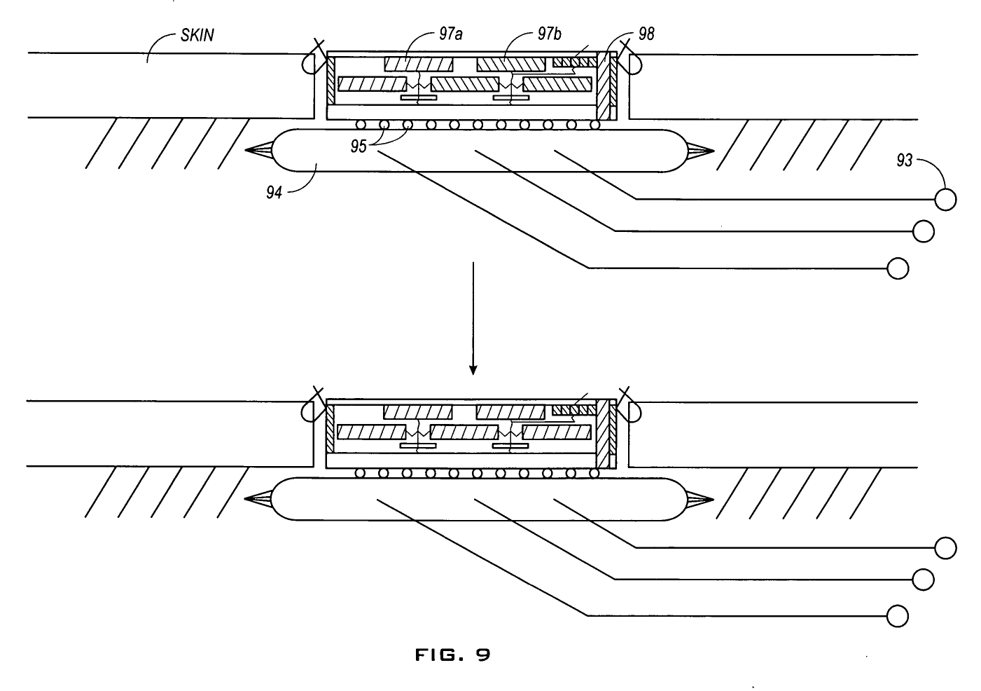

The color-EMG can also be implanted for covering a skin deficit (such as after a severe burning) for a short period of time (one or two days). Again an elastomer is used as a substrate. The color-EMG is then used as a skin transplantation that informs the patient and the medical staff about the activity status of the underlying muscle (94) and possibly also on the degree of myolysis (degeneration of the muscle). In this application the color-EMG sensing device is further provided with a reservoir (98) for the administration of fluids (physiological serum, Ringer solution, drugs, . . . ). The liquid may contain a drug for assessing the muscular function or for treating a muscular hyperactivity. FIG. 9 shows a color-EMG to replace transiently a deficit of skin and to inform the examiner and the patient on the level of muscle contraction below in order to be able to administer appropriate drugs. In FIG. 9 polymer LED (97b) shows a muscular hyperactivity, whereas LED (97a) shows a lower activity of the muscle. Note that due to the multiple electrodes (95) the color-EMG is capable of recognising a local hyperactivity of a muscle. The motoneurons (93) are innervating the muscle. By administering a myorelaxing drug via reservoir (98) the muscle will get relaxed. Optionally more than one such reservoir can be provided. By connecting a reservoir to a microcontroller capable of controlling the injection of a drug it is possible to start the drug administration when the muscular activity exceeds a given threshold value. In that way one reservoir can be used for automatic drug administration and the second reservoir for manual administration. Alternatively each reservoir can be used for a different drug. In the set-up of FIG. 6 for example a drug administration for just a certain group of axons can be provided. It is then possible to administer a drug, which can modulate the neuronal discharges in motor axons according to the level of muscle contraction.

In an alternative embodiment the color-EMG can be arranged to give an indication of the nitric oxide production in the muscle. Due to a physical effort or prolonged activity of the muscle the nitric oxide (NO) quantity in the muscle increases. The liquid comprising the NO is injected in the polymer LED. The enzyme nitrate reductase is disposed in a display. In the presence of cofactors (flavine adenine dinucleotide, cytochrome B, pterin molybdate) nitrate is transformed into nitrite. Due to the Griess reaction the nitrite is transformed into an azo compound that adopts a purple color. The more intense the purple color, the more NO is present in the liquid. A spectrophotometer can be coupled to the display in order to assess more precisely the concentration of the azo derivative. By combining this embodiment with those described previously, one can obtain a display capable of simultaneously indicating the electric activity of the muscle, the pH of the muscle as well as its NO production. When combining with the use of a color-EMG for rehabilitation purposes as previously explained, the examiner can estimate immediately if a training procedure applied in one body area affects the production of NO on another body area.

The color-EMG also proves useful for patients suffering from tension headache. Often such phenomenon occurs in a context of stress and abnormal contraction of the temporalis muscles. For such patients the color-EMG can advantageously be integrated in a pair of glasses. The temples of the glasses exert a constant pressure on the skin near the temples. In this case the pre-amplifier may be connected to the polymer LED integrated into the patient's glasses. When the muscle contracts too strongly, the person is informed so that he knows he has to relax in order reduce his headache. Such a color-EMG can advantageously have electrodes fixed to bearings (e.g. metal bearings rolling over the skin when moving). A set of suction cups can be provided to the color-EMG additionally. This allows fixing the temples to the temporalis skin. This embodiment can be combined with a bio-color-EMG, in order to assess the production of lactic acid in the temporalis muscle during tension headache episodes. The first color-EMG is located near the bio-color-EMG (on the temples of the glasses) and triggers the circulation of fluids in the bio-color-EMG when the intensity of the EMG activity is higher than a threshold. The first color-EMG can be placed on the left (or right) and the bio-color-EMG can be placed contralaterally. Moreover, the bio-color-EMG can trigger the recording of EMG activity by the first color-EMG when the level of lactic acid reaches a predetermined concentration. The bio-color-EMG can deliver simultaneously a myorelaxing drug to relax the muscle. The patient can be informed of this delivery by a signal shown on the glasses.

Certain embodiments of the color-EMG may advantageously be integrated into an intra-muscular injection needle. When a drug is to be injected in a muscle, the irritated muscle fibres are contracted instantaneously and transitorily when the needle is brought into the muscle. The color-EMG can confirm with certainty whether the extremity of the needle is well placed in the muscle. In order to keep the system low-cost, a part of the system can be disposable (cfr.supra).

Thanks to the use of the color code embodiments of such sensing devices can readily be applied to any person, even to children or elderly people. Five year old children are already capable of identifying a color code. Further it has been shown that elderly people suffering from a substantial cognitive deterioration still keep the ability to recognise colors.

While the above detailed description has shown, described, and pointed out novel features of the invention as applied to various embodiments, it will be understood that various omissions, substitutions, and changes in the form and details of the device or process illustrated may be made by those skilled in the art without departing from the spirit of the invention. As will be recognized, the present invention may be embodied within a form that does not provide all of the features and benefits set forth herein, as some features may be used or practiced separately from others.

1. A sensing device for measuring muscle activity comprising an interface with electrodes, an active amplifier, and an electronic circuit in connection with a display, said display comprising at least one semiconducting polymer LED.

2. The sensing device of claim 1, further comprising at least one membrane and means for circulating a liquid connected to said membrane.

3. The sensing device of claim 1, wherein the active amplifier is provided with a battery.

4. The sensing device of claim 1, wherein the electronic circuit further comprises a passive filter.

5. The sensing device of claim 1, wherein said semiconducting polymer LED comprises a substrate, an epoxy membrane, an anode, a conducting polymer, an emissive polymer layer and a cathode.

6. The sensing device of claim 5, wherein said substrate comprises an elastomer.

7. The sensing device of claim 1, comprising at least two polymer LEDs and wherein an inverter is arranged between the electronic circuit and one of the polymer LEDs.

8. The sensing device of claim 1, wherein an analogue converter is arranged between the electronic circuit and the display.

9. The sensing device of claim 1, wherein the interface comprises fine wire electrodes.

10. The sensing device of claim 1, which is portable by a patient.

11. The sensing device of claim 1, wherein the connection between said electronic circuit and said display comprises a wireless connection.

12. The sensing device of claim 1, further comprising an electrode for measuring nerve activity.

13. The sensing device of claim 1, comprising a stack of polymers LEDs, two consecutive polymer LEDs of said stack being separated from each other with a substrate arranged for preventing light to pass.

14. The sensing device of claim 1, further comprising a waterproof covering.

15. The sensing device of claim 1, comprising a plurality of electrodes, said electrodes being positioned in such arrangement that information on the direction of muscular activity is derivable.

16. The sensing device of claim 1, further comprising at least one reservoir.

17. The sensing device of claim 1, wherein said polymer LED comprises an enzyme for detecting nitric oxyde.

18. The sensing device of claim 17, wherein said enzyme is nitrate reductase.

19. The sensing device of claim 1, wherein said electrodes are fixed to bearings.

20. An injection needle, comprising an interface with electrodes, an active amplifier, and an electronic circuit in connection with a display, said display comprising at least one semiconducting polymer LED.

21. A method of measuring muscle activity of a patient comprising the acts of maintaining an interface with electrodes upon the patient's skin for a sufficient time to obtain a measure of muscle activity of the patient, wherein said interface with electrodes is in connection with an active amplifier, an electronic circuit and a display comprising at least one semiconducting polymer LED.

22. The method of claim 21, additionally comprising recording the muscle activity of the patient.

23. The method of claim 21, additionally comprising diagnosing a symptom or a disease selected from the group consisting of torticolis, neuro-degenerative diseases, muscles over-activation or under-activation, primary orthostatic tremor (POT) or myoclonus.

24. The method of claim 21, additionally comprising monitoring of a muscle rehabilitation of the patient.

25. The method of claim 23, wherein the monitoring of muscle rehabilitation is performed following grafting.

26. The method of claim 21, additionally comprising analyzing the intramuscular pH of a muscle, analyzing of muscle NO production, or administrating a fluid or a drug to a muscle.
